(19) United States
(12) Patent Application Publication
(10) Pub. No.: US 2003/0035851 A1
(43) Pub. Date: Feb. 20, 2003

(54) Anti-cancer agents and method of use thereof

(76) Inventor: Sophie Chen, Millwood, NY (US)

Correspondence Address: CANTOR COLBURN, LLP, 55 GRIFFIN ROAD SOUTH, BLOOMFIELD, CT, 06002

(21) Appl. No.: 10/072,823

(22) Filed: Feb. 8, 2002

Related U.S. Application Data
(60) Provisional application No. 60/267,331, filed on Feb. 8, 2001.
(60) Provisional application No. 60/308,213, filed on Jul. 27, 2001.

(51) Int. Cl. A61K 35/78

(52) U.S. Cl. 424/728

(57) ABSTRACT

A composition effective in suppressing the growth of cancer cells comprises a compound selected from the group consisting of oridonin, lupulone, bavachin, bavachalcone, bavachinin, bavachromene, their pharmaceutically acceptable salts or esters, their selectively substituted analogs, and a combination comprising at least one of the foregoing. Another embodiment is an improved method for the treatment of various cancers, comprising administration of a pharmaceutically effective quantity of a compound selected from the group consisting of oridonin, lupulone, bavachin, bavachalcone, bavachinin, bavachromene, their pharmaceutically acceptable salts or esters, their selectively substituted analogs, and a combination comprising at least one of the foregoing.

### CROSS-REFERENCE TO RELATED APPLICATIONS

This application claims priority to U.S. Application Serial No. 60/267,331, filed Feb. 8, 2000, and U.S. Application Serial No. 60/308,213, filed Jul. 27, 2000, the contents of both applications being incorporated herein in their entirety.

### BACKGROUND OF THE INVENTION

Carcinomas, such as prostate and breast cancers, are a major health problem among men and women in North America and Europe. Every year 160,000 new cases and 39,000 deaths from the disease occur in the U.S. (Landis, S. H. et al, “Cancer Statistics, 1998”, CA Cancer J. Clin., Volume 48, pages 6-29 (1998)); and new invasive incidences of breast carcinoma are projected to be 192,200, with 40,200 projected deaths in 2001 according to the American Cancer Society (National Alliance of Breast Cancer Organizations News, Vol. 15, No. 1, page 2, January, 2001). Early detection and early intervention are the key solution to these diseases. Conventional treatment methods include surgery, radiation, hormone therapy, and chemotherapy. Although chemotherapy is the choice for advanced-stage breast cancer patients, it is not effective for the advanced-stage prostate cancer patients. Therefore, there is a need for alternative therapeutic agents that can augment or replace existing therapies.

Recently, an herbal supplement, PC SPES, has been used among some prostate cancer patients (see J. Lewis, Jr. and E. R. Berger, New Guidelines for Surviving Prostate Cancer, Health Education Literary Publisher, Westbury, N.Y. (1997)). PC SPES is composed of the extracts from eight herbs, namely Dendranthema morifolium; Ganoderma lucidium; Glycyrrhiza uraensis; Isatis indigotica; Panax pseudo-ginseng; Rabdosia rubescens; Scutellaria baicalensis; and Serenoa repens. While the clinical benefit awaits future rigorous investigation, preliminary clinical studies (phase I and II) suggest that PC SPES is a well-tolerated and active treatment for androgen-independent prostate cancer patients. To further verify the safety and efficacy of PC SPES, the National Institute of Health (NIH) has funded clinical study. Therefore, it is highly desirable to investigate the molecular action of PC SPES based on its active components.

Recent research has also focused on the anti-cancer effects of oridonin, a compound found in the Rabdosia rubescens component of PC SPES. Studies on the anti-cancer effect of oridonin suggest that oridonin inhibits leukemic cell growth. This anti-leukemic activity has been attributed to the inhibitory effect on deoxyribonucleotide (DNA) and (ribonucleotide) RNA synthesis, and on the metabolism of thymine nucleotides in leukemic cells. However, there has been no report suggesting that oridonin is inhibitory to prostate cancer cell growth, or to other cancer cell growth.

Oridonin is a diterpene isolated from Rabdosia rubescens, which is a Chinese herb sometimes used to treat esophageal cancer (Kee Chang Huang, editor, The Pharmacology of Chinese Herbs, CRC Press, Inc., 1993, page 352). The structure of oridonin (CAS Registry No. 28957-04-2) is given below:

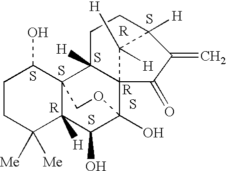

Another organic compound that poses some interest as to its inhibitory effect on cancer cells is lupulone. Lupulone is a bitter substance purified from hops, or Humulus lupulus L. (Beijing Traditional Chinese Medical College, editor, “Chinese Herbal Medicinal Chemistry”, Shanghai Technology Publisher, 1974, page 215). The structure of lupulone (CAS Registry No. 35049-52-6) is given below:

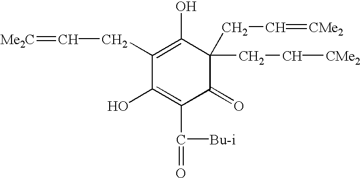

Little has been published about lupulone's biological activity. Tagashira et al. observed that lupulone exhibits anti-lipid peroxidation activity, but not anti-free radical activity (M. Tagashira, M. Watanabe, and N. Uemitsu, “Antioxidative Activity Of Hop Bitter Acids And Their Analogues”, Biosci. Biotechnol. Biochem., Vol. 59, No. 4, pages 740-2 (1995)). None of the literature suggests that lupulone possesses any one or more of anti-prostate cancer, anti-breast cancer, anti-liver cancer, anti-bone cancer, or anti-bladder cancer activity.

Despite the advances made in understanding oridonin and lupulone, still more discovery is needed as to the types of cancer cells they can inhibit and as to the mechanisms by which they can control cell proliferation. Contributing to the success of finding adequate cancer treatments, is the investigation as to other natural chemical compounds capable of acting in tandem and separately with oridonin and lupulone; many of such compounds may come from the investigation of herbal compounds known to have medicinal effects.

### BRIEF SUMMARY OF THE INVENTION

A composition effective in suppressing the growth of cancer cells comprises a compound selected from the group consisting of oridonin, lupulone, bavachin, bavachalcone, bavachinin, bavachromene, their pharmaceutically acceptable salts or esters, their selectively substituted analogs, and a combination comprising at least one of the foregoing.

Another embodiment is an improved method for the treatment of various cancers, comprising administration of a pharmaceutically effective quantity of a compound selected from the group consisting of oridonin, lupulone, bavachin, bavachalcone, bavachinin, bavachromene, their pharmaceutically acceptable salts or esters, their selectively substituted analogs, and a combination comprising at least one of the foregoing.

### BRIEF DESCRIPTION OF THE DRAWINGS

FIG. 1 is a plot of growth inhibition of DU-145 and LNCaP prostate cancer cells as a function of oridonin concentration.

FIG. 2 is a plot of growth inhibition of DU-145 and LNCaP prostate cancer cells as a function of lupulone concentration.

FIG. 3 is a plot of growth inhibition of MCF-7 breast cancer cells as a function of oridonin and lupulone concentrations.

FIG. 4 is two DNA histograms showing the effect on the LNCaP cell cycle in the absence of oridonin (“CTRL”) and in the presence of oridonin at 3 microgram/milliliter.

FIG. 5 is two bar graphs illustrating the differential effect on the LNCaP cell cycle where the first graph shows the effect in the absence of oridonin (“CTRL”) and in the presence of 3 μg of oridonin, and the first graphs shows the effect in the absence of lupulone (“CTRL”) and in the presence of 50 μg of lupulone.

FIG. 6 is a bar graph illustrating the differential effect on the LNCaP cell cycle in the absence of oridonin (“CTRL”) and in the presence of 1.5 μg/ml and 3 μg/ml of oridonin.

FIG. 7 is a bar graph illustrating the differential effect on the MCF-7 cell cycle in the absence of lupulone (“CTRL”) and in the presence of 25 μg/ml and 50 μg/ml of lupulone.

FIG. 8 is a representation of the data obtained when a sample of oridonin was exposed to HPLC at a reaction time of 26.8 minutes.

FIG. 9 is 4 DNA histograms showing the effect on the LNCaP (a-b) and the DU-145 (c-d) cell cycles in the absence of oridonin (“CTRL”) (a and c) and in the presence of 13.74 mM of oridonin (b and d).

FIG. 10 is two bar graphs illustrating the dose-responsive effect on the LNCaP and DU-145 cell cycles in the absence of oridonin and in the presence of varying concentrations of oridonin.

FIG. 11 is a Western blot illustrating Bax and Bcl-2 protein expression in the absence of oridonin (“CTRL”) and in the presence of varying concentrations of oridonin.

FIG. 12 is a bar graph illustrating the effect in LNCaP on p53 protein expression in the absence of oridonin (“CTRL”) and in the presence of varying concentrations of oridonin.

FIG. 13 is a representation of apoptosis as shown by TdT staining wherein the first graph shows apoptosis in the absence of oridonin (“CTRL”), the second graph shows apoptosis in the presence of 8.24 mM of oridonin, and the third graph shows apoptosis in the presence of 13.74 mM of oridonin.

FIG. 14 is a plot illustrating the cell growth inhibition of LNCaP and Du-145 in the presence of varying concentrations of DES.

FIG. 15 is a bar graph illustrating the differential effect on the LNCaP cell cycle in the absence of PC SPES and DES (“CTRL”) and in the presence of 2 μl/ml of PC SPES and 30 μM of DES.

FIG. 16 is a ¹³C NMR spectrum of oridonin.

FIG. 17 is a DEPT correlation spectrum of oridonin.

FIG. 18 is an EI mass spectrum of oridonin.

### DETAILED DESCRIPTION OF THE PREFERRED EMBODIMENTS

Disclosed herein is a composition for treating and/or preventing various forms of cancer, such as prostate, breast, colon, lung, and bladder cancers. More specifically, the composition comprises compounds from various plant sources. These compounds may be extracts found naturally in the plant, or they may be synthesized and/or altered by pharmaceutical means. The plant sources may include, but are not limited to, Rabdosia rubescens, Humulus lupulus, Psoralea corylifolia L, Panax pseudo-ginseng Wall, Ganoderma lucidum Karst, Scutellaria baicalensis Georgi, Glycine max, Curcuma longa, and combinations comprising at least one of the foregoing plants.

The extracts include, but are not limited to, oridonin, lupulone, bavachin, bavachalcone, bavachinin, bavachromene, gensenoside, baicalin, soy flavonoid, soy isoflavonoid, curcumin, pharmaceutically acceptable salts or esters, selectively substituted analogs, and combinations comprising at least one of the foregoing extracts.

Generally, oridonin is an extract of Rabdosia rubescens; lupulone is an extract of Humulus lupulus; bavachin, bavachalcone, bavachinin, and bavachromene are extracts of Psoralea corylifolia L.; gensenoside is an extract of Panax pseudo-ginseng Wall; baicalin is an extract of Scutellaria baicalensis Georgi; soy flavonoid and soy isoflavonoid are extracts of Glycine max; and curcummin is an extract of Curcuma longa. However, it is contemplated that these extracts may also be found in other biological organisms.

A first embodiment is a composition effective in suppressing the growth of cancer cells comprising a compound selected from the group consisting of oridonin, lupulone, bavachin, bavachalcone, bavachinin, bavachromene, their pharmaceutically acceptable salts or esters, their selectively substituted analogs, and a combination comprising at least one of the foregoing.

The composition may comprise oridonin, its pharmaceutically acceptable salts or esters, its selectively substituted analogs, or a combination comprising at least one of the foregoing. In one embodiment, the composition may comprise a compound having the structure

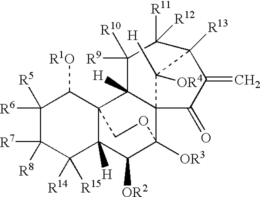

wherein R¹-R⁴ are each independently hydrogen, C₁-C₆ alkyl, or C₁-C₁₂ acyl; R⁵-R¹³ are each independently hydrogen or C₁-C₆ alkyl, with the proviso that at least 4 of R⁵-R¹³ are hydrogen; and R¹⁴ and R⁵ are each independently C₁-C₆ alkyl. In a preferred embodiment R¹-R⁴ are each independently hydrogen, methyl, ethyl, acetyl, or propionyl. In another preferred embodiment, R⁵-R¹³ are each independently hydrogen, methyl, or ethyl. In a highly preferred embodiment, R¹-R¹³ are hydrogen, and R¹⁴ and R¹⁵ are methyl. The composition may comprise an extract of Rabdosia rubescens comprising oridonin.

The composition may comprise lupulone, its pharmaceutically acceptable salts or esters, its selectively substituted analogs, or a combination comprising at least one of the foregoing. In one embodiment, the composition may comprise a compound having the structure

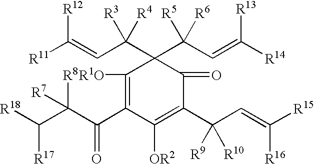

wherein R¹ and R² are each independently hydrogen, C₁-C₆ alkyl, or C₁-C₁₂ acyl; R³-R¹⁰ are each independently hydrogen or C₁-C₆ alkyl with the proviso that at least four of R³-R¹⁰ are hydrogen; and R¹¹-R¹⁸ are each independently C₁-C₆ alkyl. In a preferred embodiment, R¹ and R² are each independently hydrogen, methyl, ethyl, acetyl, or propionyl. In another preferred embodiment, R³-R¹⁰ are each independently hydrogen, methyl, or ethyl. In another preferred embodiment, R¹¹-R¹⁸ are each independently methyl or ethyl. In a highly preferred embodiment, R¹-R¹⁰ are each hydrogen and R¹¹-R¹⁵ are each methyl. The composition may comprise an extract of Humulus lupulus comprising lupulone.

The composition may comprise bavachin or bavachinin, their pharmaceutically acceptable salts or esters, their selectively substituted analogs, or a combination comprising at least one of the foregoing. In one embodiment, the composition may comprise a compound having the structure

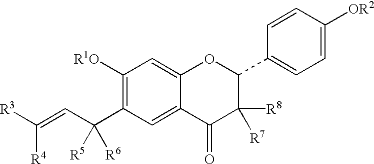

wherein R¹ and R² are each independently hydrogen, C₁-C₆ alkyl, or C₁-C₁₂ acyl; and R³-R⁸ are each independently hydrogen or C₁-C₆ alkyl with the proviso that at least two of R³-R⁸ are hydrogen. In a preferred embodiment, R¹ and R² are each independently hydrogen, methyl, ethyl, acetyl, or propionyl. In another preferred embodiment, R³-R⁸ are each independently hydrogen, methyl, or ethyl. In another preferred embodiment, R³ and R⁴ are methyl. In a highly preferred embodiment, R¹, R², and R⁵-R⁸ are hydrogen; and R³ and R⁴ are methyl. In another highly preferred embodiment, R² and R⁵-R⁸ are hydrogen; and R¹, R³, and R⁴ are methyl. The composition may comprise an extract of Psoralea corylifolia L. comprising bavachin. The composition may comprise an extract of Psoralea corylifolia L. comprising bavachinin.

The composition may comprise bavachalcone, its pharmaceutically acceptable salts or esters, its selectively substituted analogs, or a combination comprising at least one of the foregoing. In one embodiment, the composition may comprise a compound having the structure

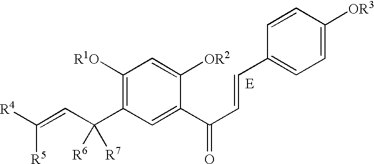

wherein R¹-R³ are each independently hydrogen, C₁-C₆ alkyl, or C₁-C₁₂ acyl; and R⁴-R⁷ are each independently hydrogen or C₁-C₆ alkyl. In a preferred embodiment, R¹-R³ are each independently hydrogen, methyl, ethyl, acetyl, or propionyl. In another preferred embodiment, R⁴-R⁷ are each independently hydrogen, methyl, or ethyl. In a highly preferred embodiment, R¹-R³, R⁶, and R⁷ are hydrogen; and R⁴ and R⁵ are methyl. The composition may comprise an extract of Psoralea corylifolia L. comprising bavachalcone.

The composition may comprise bavachromene, its pharmaceutically acceptable salts or esters, its selectively substituted analogs, or a combination comprising at least one of the foregoing. In one embodiment, the composition may comprise a compound having the structure

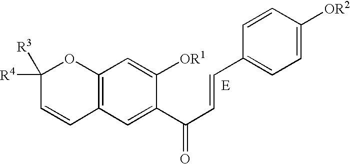

wherein R¹ and R² are each independently hydrogen, C₁-C₆ alkyl, or C₁-C₁₂ acyl; and R³ and R⁴ are each independently hydrogen or C₁-C₆ alkyl. In a preferred embodiment, R¹ and R² are each independently hydrogen, methyl, ethyl, acetyl, or propionyl. In another preferred embodiment, R³ and R⁴ are each independently hydrogen, methyl, or ethyl. In a highly preferred embodiment, R¹ and R² are hydrogen; and R³ and R⁴ are methyl. The composition may comprise an extract of Psoralea corylifolia L. comprising bavachromene.

Oridonin, lupulone, bavachin, bavachalcone, bavachinin, bavachromene, their pharmaceutically acceptable salts or esters, or their selectively substituted analogs may be isolated from natural sources or synthesized according to known methods. Purities of these compounds, as employed in the composition, may vary according to their method of isolation or synthesis, but purities of about 5 percent to greater than 99 percent may be suitable for use in the composition.

An exemplary composition for the treatment and/or the prevention of certain cancers, such as prostate, breast, colon, lung, bladder, and the like comprises combining a Rabdosia rubescens extract, such as oridonin, or a Humulus lupulus extract, such as lupulone, with an extract taken from a Panax pseudo-ginseng Wall, wherein the extract is preferably gensenoside; a Ganoderma lucidum Karst extract; a Scutellaria baicalensis Georgi extract, wherein the extract is preferably baicalin; a Glycine max extract, wherein the extract is preferably a soy flavonoid, a soy isoflavonoid, or a combination of both; and a Curcuma longa extract, wherein the extract comprises curcumin.

Here, the composition comprises about 1-20 parts by weight (pbw) of the Rabdosia rubescens or the Humulus lupulus, where about 1-15 pbw is particularly preferred, and about 2-10 pbw is more preferred; about 5-60 pbw of the Panax pseudo-ginseng Wall extract, where about 8-50 pbw is more particularly, and about 10-40 is more preferred; about 50-600 pbw of the Ganoderma lucidum Karst extract, where about 80-555 pbw is particularly preferred, and about 100-500 pbw is more preferred; about 10-120 pbw of the Scutellaria baicalensis Georgi extract, where about 15-110 pbw is particularly preferred, and about 20-100 pbw is more preferred; about 10-120 pbw of the Glycine max extract, where about 15-110 pbw is particularly preferred, and about 20-100 pbw is more preferred; and about 12-120 pbw of the curcuma longa extract, where about 15-100 pbw is particularly preferred, and about 20-100 pbw is more preferred. Here pbw is defined as the individual weight of each component in proportion to the total weight of the composition.

A second embodiment is an improved method for the treatment of various cancers, comprising administration of a pharmaceutically effective quantity of a compound selected from the group consisting of oridonin, lupulone, bavachin, bavachalcone, bavachinin, bavachromene, their pharmaceutically acceptable salts or esters, their selectively substituted analogs, and a combination comprising at least one of the foregoing. Based on the data presented below, such administration is effective to have anti-prostate cancer, anti-breast cancer, anti-colon cancer, anti-lung cancer, or anti-bladder cancer activity in vivo.

Methods for the formulation of pharmaceutically acceptable compositions are generally known. The subject pharmaceutical formulations may comprise one or more non-biologically active compounds, i.e., excipients, such as stabilizers (to promote long term storage), emulsifiers, binding agents, thickening agents, salts, preservatives, and the like, depending on the route of administration.

For oral administration, the oridonin, lupulone, bavachin, bavachalcone, bavachinin, bavachromene, their pharmaceutically acceptable salts or esters, their selectively substituted analogs, and a combination comprising at least one of the foregoing may be administered with an inert diluent or with an assimilable edible carrier, or incorporated directly with the food of the diet. The formulations may be incorporated with excipients and used in the form of ingestible tablets, buccal tablets, troches, capsules, elixirs, suspension syrups, wafers, and the like. The tablets, troches, pills, capsules and the like may also contain the following: a binder, such as gum tragacanth, acacia, cornstarch, or gelatin; excipients, such as dicalcium phosphate; a disintegrating agent such as corn starch, potato starch, alginic acid and the like; a lubricant such as magnesium stearate; a sweetening agent, such as sucrose, lactose or saccharin; and a flavoring agent such as peppermint, oil of wintergreen, or the like flavoring. When the dosage unit is a capsule, it may contain, in addition to materials of the above type, a liquid carrier. Various other materials may also be present as coatings or to otherwise modify the physical form of the dosage unit. A syrup or elixir may contain sucrose as a sweetening agent, methyl and propylparabens as preservatives, a dye and flavoring such as cherry or orange flavor. Such additional materials should be substantially non-toxic in the amounts employed. Furthermore, the active agents may be incorporated into sustained-release preparations and formulations. Formulations for parenteral administration may include sterile aqueous solutions or dispersions, and sterile powders for the extemporaneous preparation of sterile, injectable solutions or dispersions. The solutions or dispersions may also contain buffers, diluents, and other suitable additives, and may be designed to promote the cellular uptake of the active agents in the composition, e.g., liposomes. Sterile injectable solutions are prepared by incorporating the active compounds in the required amount in the appropriate solvent with one or more of the various other ingredients described above, followed by sterilization. Dispersions may generally be prepared by incorporating the various sterilized active ingredients into a sterile vehicle that contains the basic dispersion medium and the required other ingredients from those listed above. In the case of sterile powders used to prepare sterile, injectable solutions, the preferred methods of preparation are vacuum-drying and freeze-drying techniques which yield a powder of the active ingredient plus any additional desired ingredient from previously sterile-filtered solutions. Pharmaceutical formulations for topical administration may be especially useful for localized treatment. Formulations for topical treatment included ointments, sprays, gels, suspensions, lotions, creams, and the like. Formulations for topical administration may include known carrier materials such as isopropanol, glycerol, paraffin, stearyl alcohol, polyethylene glycol, and the like. The pharmaceutically acceptable carrier may also include a known chemical absorption promoter. Examples of absorption promoters are e.g., dimethylacetamide (U.S. Pat. No. 3,472,931), trichloroethanol or trifluoroethanol (U.S. Pat. No. 3,891,757), certain alcohols and mixtures thereof (British Patent No. 1,001,949), and British Patent No. 1,464,975. Except insofar as any conventional media or agent is incompatible with the therapeutic active ingredients, its use in the therapeutic compositions and preparations is contemplated.

Supplementary active ingredients can also be incorporated into the compositions and preparations. For example, administration of oridonin, lupulone, bavachin, bavachalcone, bavachinin, bavachromene, their pharmaceutically acceptable salts or esters, their selectively substituted analogs, or a combination comprising at least one of the foregoing in combination with other anti-cancer agents is expected to maximally stimulate anti-cancer activity.

The compositions and preparations described preferably contain at least 0.1% of active agent. The percentage of the compositions and preparations may, of course, be varied and may contain between about 2% and 60% of the weight of the amount administered. The amount of active compounds in such pharmaceutically useful compositions and preparations is such that a suitable dosage will be obtained.

The invention is further illustrated by the following non-limiting examples.

General Experimental Approach

The following examples set out to show the inhibitory effect of herbal extracts, oridonin and lupulone, on the proliferation of various cancer cell lines, wherein proliferation is defined as the cell's ability to mitotically divide. The examples also show the relationship between oridonin, and the expression of tumor suppressor genes, p53 and Bax, which are also pro-apoptotic genes, and the expression of tumor promoter gene, Bcl-2, which is an anti-apoptotic gene.

### EXAMPLE 1

### Isolating and characterizing oridonin from Rabdosia rubescens

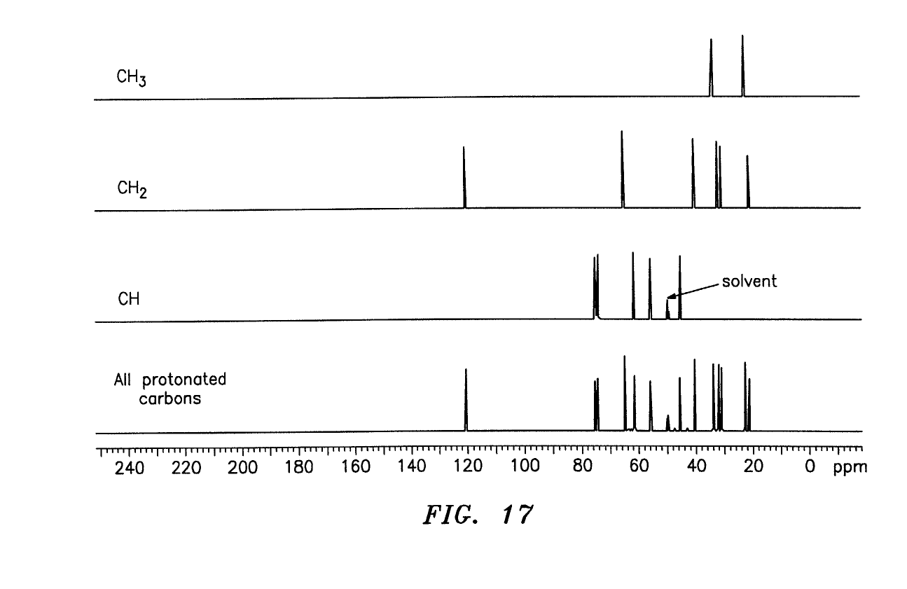

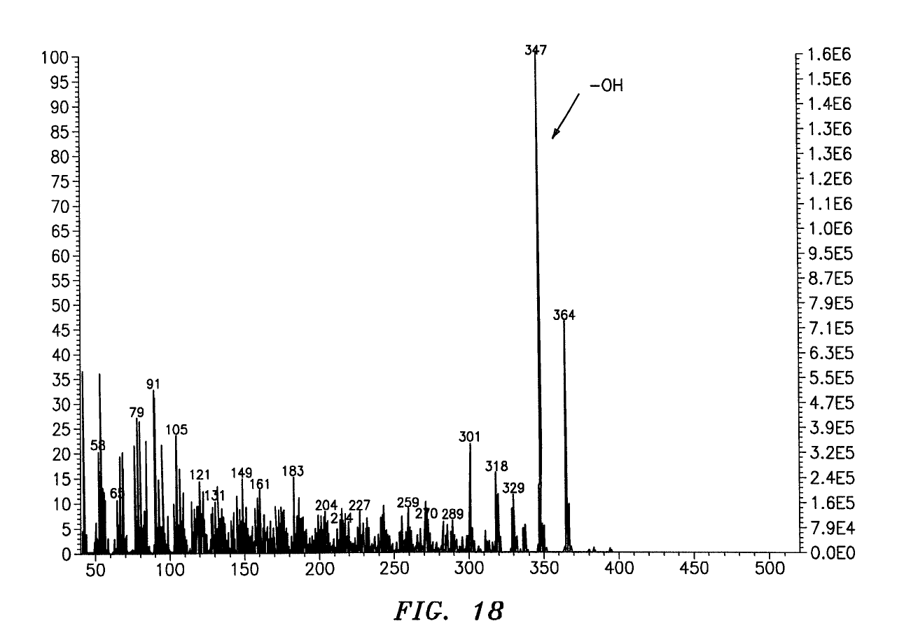

Oridonin was isolated and purified from Rabdosia rubescens according to the method of K. Yuan et al. (K. Yuan, R. Hu, C. Ji, and M. Yin, “New Method For Preparing Oridonin By Column Chromatography”, Chung Kuo Chung Yao Tsa Chih, volume 22, no. 8, pages 478-80, 511 (1997)). The isolated compound was further recrystallized from ethanol. The purity of oridonin isolated from Rabdosia rubescens was confirmed by High Performance Liquid Chromatography (HPLC) to be greater than about 95% as shown in FIG. 8. The purified oridonin product's chemical structure was determined by ¹³C NMR (nuclear magnetic resonance spectroscopy) spectrum as shown in FIG. 16, and DEPT (distorionless enhancement by polarization transfer) correlation spectra as shown in FIG. 17. The assignment of chemical shifts of oridonin is presented in Table 1.

TABLE 1
¹³C NMR data of oridonin

| C | δ ppm | DEPT | C | δ ppm | DEPT |
|---|---|---|---|---|---|
| 1 | 74.9* | CH | 11 | 20.8 | CH₂ |
| 2 | 30.5 | CH₂ | 12 | 31.5 | CH₂ |
| 3 | 39.8 | CH₂ | 13 | 55.2 | CH |
| 4 | 34.6 | C | 14 | 74.3* | CH |
| 5 | 44.8 | CH | 15 | 209 | C |
| 6 | 73.9 | CH | 16 | 153.2 | C |
| 7 | 98.4 | C | 17 | 120.4 | CH₂ |
| 8 | 63.1 | C | 18 | 33.2 | CH₃ |
| 9 | 61.1 | CH | 19 | 22.1 | CH₃ |
| 10 | 42.4 | CH | 20 | 64.5 | CH₂ |

*may be exchanged

The molecular weight was determined by high energy electron impact (EI) mass spectra to be 364 as shown in FIG. 18.

Lupulone was isolated from Humulus lupulus according to the procedure of Shiao (C. H. Shiao, “Chinese Herbal Medicinal Chemistry”, Shanghai Technology Publisher, page 402 (1987)). The anti-cancer activities of oridonin and lupulone were evaluated by determining their abilities to inhibit cancer cell growth, to modulate cancer cell cycle, to induce cell apoptosis, and to regulate hormone and cytokine receptors (T. Hsieh, S. S. Chen, X. Wang, and J. Wu, “Regulation of Androgen Receptor (AR) and Prostate Specific Antigen (PSA) in the Androgen-Responsive Human Prostate LNCaP Cells by Ethanolic Extracts of the Chinese Herbal Preparation PC-SPES.” Biochem. Mol. Biol. Int., volume 42, pages 535-544 (1997); S. Chen, Q. Ruan, E. Bedner, A. Beptala, X. Wang, T. C. Hsieh, F. Traganos, and Z. Darzynkiewicz, “Effects of the Flavonoid Baicalin and its Metabolite Baicalein on Androgen Receptor Expression, Cell Cycle Progression and Apoptosis of Prostate Cancer Cell Lines”, Cell Proliferation, in press (2001); H. D. Halicka, B. Ardelt, G. Juan, A. Mittelman, S. Chen, F. Traganos and Z. Darzynkiewicz, “Apoptosis and Cell Cycle Effects Induced by Extracts of the Chinese Herbal Preparation of PC SPES”, International J. of Oncology, volume 11, pages 437-448 (1997)).

### EXAMPLE 2

### Preparing the Cell Cultures

Cancer cell lines LNCaP, DU-145, and MCF-7 cells were purchased from American Type Culture Collection and maintained in RPMI 1640 culture media supplemented with 10% heat-inactivated fetal bovine serum (FBS), 2 millimolar (Mm) L-glutamine, 100 units/milliliter of penicillin, and 100 grams/milliliter (g/ml) of streptomycin at 37.5° C. in an atmosphere of 5% carbon dioxide (CO₂) in air. The cells were routinely seeded at 1×10⁵ cells/ml in T-75 flasks, allowed to attach overnight, and then treated with the oridonin or the lupulone. At different times, the cells were harvested by trypsinization.

LNCaP is an androgen receptor positive prostate cancer cell line; MCF-7 is an androgen receptor positive breast cancer cell line; and DU-145 is an androgen receptor negative prostate cancer cell line that is very difficult to inhibit with known drugs, thereby making DU-145 an excellent model to be used to study the efficacy of the anti-cancer agents.

### EXAMPLE 3

### The inhibitory effect of oridonin and lupulone on the proliferation of LNCaP, DU-145, and MCF-7 cell lines.

The inhibitory effect on a cell's ability to divide was determined by an MTT (3-(4,5-dimethylthiazol-2-yl)-2,5-diphenyl tetrazolium bromide) assay. The assay reagents were purchased from Boehringer Mannheim (Roche Diagnosis Corp, Indianapolis, Ind.). MTT is cleaved to form formazan by metabolically active cells and exhibits a strong red absorption band at 550-618 nanometers (nm). The MTT assay provides a method whereby cell proliferation can be determined by counting cells. The protocol for the cell viability assay was provided by Boehringer Mannheim and modified as described below.

LNCaP, DU-145 and MCF-7 cells were separately seeded in 96 well microtiter plates at a concentration of 3×10³ MCF-7 cells per well, and 6×10³ LNCaP cells per well in a volume of 100 microliters of cell culture medium. The cells were then incubated at ______ and allowed to attach to the plates. After 24 hours, 20 microliter aliquots having varying concentrations of oridonin or lupulone were added to the cell culture. Each aliquot was plated into 3 wells to obtain mean values. To eliminate any solvent effect, 20 microliters of the solvent used in the preparation of the highest concentration of the oridonin or the lupulone (a maximum of 0.5% by volume of dimethylsulfoxide (DMSO)) was added to the wells containing LNCaP or MCF-7 cells, wherein no oridonin or lupulone had been added (Control cells). The plates were incubated at 37° C. in a CO₂ incubator for 72 hours. After 72 hours, the culture medium was carefully removed without disturbing the cells and replaced by 100 microliters of fresh cell medium. 10 microliters of MTT was added to each well and the plates were incubated in the CO₂ incubator at 37° C. for 4 hours. Afterwards, 100 microliters of sodium dodecylsulfate (SDS) solubilizing reagent (from Boehringer Mannheim) was added to each well. The plates were placed in the CO₂ incubator at 37° C. for about 10-14 hours. Afterwards, the cell concentration of each plate was determined by an ELISA Reader (EL800, Bio-Tek Instruments, Inc.) at a wavelength of 570 μm. The percent cell viability was calculated according to the equation below:

$$V = 100 ( \frac{{A}_{control} - {A}_{treated}}{{A}_{control}} )$$

where V is the percent cell viability, A_{control} is the absorption of the control cells, and A_{treated} is the absorption of the treated cells.

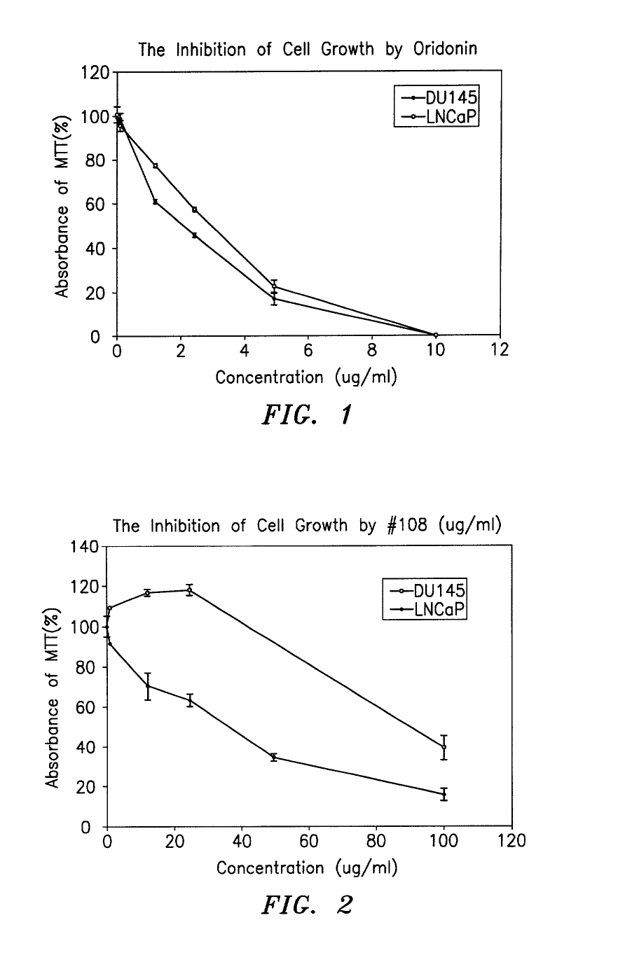

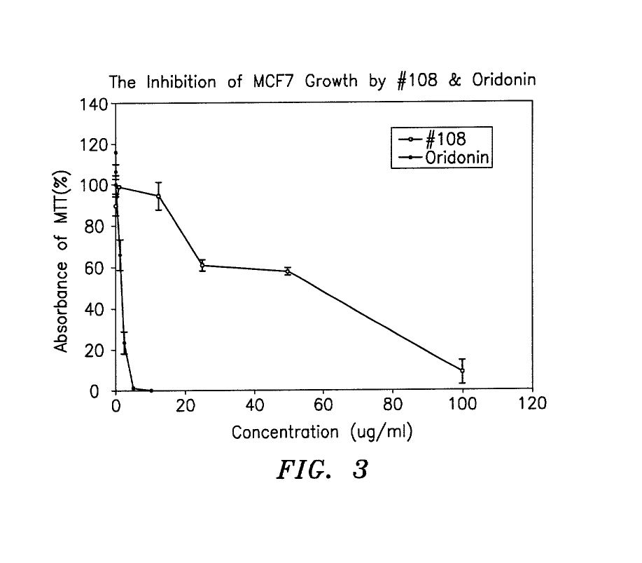

FIGS. 1, 2, and 3 demonstrate that both oridonin and lupulone are effective growth inhibitors of both androgen receptor positive cell lines (LNCaP and MCF-7 cell lines), and of androgen receptor negative cell lines (DU-145). For example, FIG. 1 shows that at concentrations of about 5 ug/ml of oridonin, the proliferation of more than about 75% of the LNCaP cells is reduced by about 75% over that of the control, and the proliferation of DU-145 cells is decreased by more than about 80% over that of the control. FIG. 2 shows that at concentrations of about 100 ug/ml of lupulone, the proliferation of DU-145 cells is decreased by more than about 55% compared to the control, and the proliferation of LNCaP is decreased by more than about 75% compared to the control. FIG. 3, indicates that MCF-7 cells also exhibit growth inhibition as a result of exposure to oridonin and lupulone. For example, at concentrations of under 15 ug/ml of oridonin, the absorbance reading of MTT is 0; thereby indicating that there are no metabolically active cells in the sample which suggests that there is no cell proliferation occurring in the sample. At concentrations of about 100 ug/ml of lupulone, the proliferation of MCF-7 cells is decreased by more than about 85% compared to the control.

Based on FIGS. 1-3, it is apparent that the inhibition of cell growth is dose dependent. The concentration of the compounds resulting in 50% inhibition of cancer cell growth, defined as ED₅₀, was determined by linear interpolation of FIGS. 1-3. ED₅₀ values for the three compounds obtained from these measurements are shown in Table 2.

TABLE 2
ED50 values from MTT Assay as a Function of Compound and Cell
Type

| | LNCaP | DU-145 | MCF-7 |
|---|---|---|---|
| Oridonin | 2.11 μg/ml | 3.03 μg/ml | 1.71 μg/ml |
| Lupulone | 36.53 μg/ml | 89.7 μg/ml | 56.62 μg/ml |

### EXAMPLE 4

### The Effect of Oridonin and Lupulone on LNCaP Cell Cycles

The cell cycle is the program for cell growth and cell proliferation (cell division). There are four phases of the cell cycle: G1, S, G2, and M. The G1 phase is characterized by gene expression and protein synthesis, and is regulated primarily by extracellular stimuli. During the S phase, the cell replicates its DNA. During the G2 phase, the cell undergoes growth and protein synthesis in preparation for cell division. The M phase is characterized by cytokinesis of the cell into two daughter cells.

The effect of oridonin and lupulone on LNCaP cells was determined by the following method. 2×10⁶-4×10⁶ cultured cells were exposed to varying concentrations (e.g., 1.5 microgram/milliliter (ug/ml)-3 ug/ml) of oridonin or lupulone for 24-48 hours in 12.5 cm² area flasks before being harvested. The cells were washed with phosphate buffered saline (PBS) and fixed in ice-cold 70% ethanol. Aliquots of fixed cells were rehydrated in PBS and stained with 1.0 μg/ml of 4,6-diamidino-2-phenylindole (DAPI) (Eastman Kodak, Rochester, N.Y.) dissolved in 10 mM piperazine-N,N-bis-2-ethane-sulfonic acid buffer (Calbiochem, La Jolla, Calif.) containing 100 mM NaCl, 2 mM MgCl₂ and 0.1% Triton X-100 (Sigma) at pH 6.8 as previously described by Halicka et al. (H. D. Halicka, B. Ardelt, G. Juan, A. Mittelman, S. Chen, F. Traganos and Z. Darzynkiewicz, “Apoptosis and Cell Cycle Effects Induced by Extracts of the Chinese Herbal Preparation of PC SPES”, International J. of Oncology, volume 11, pages 437-448 (1997)).

The distribution of cells in various stages of the cell cycle were determined by first staining the cells with the DNA specific fluorochrome, and then by measuring cellular DNA content with an ELITE ESP flow cytometer (Coulter Inc., Fl.) using UV laser illumination. The Multicycle program (Phoenix Flow Systems) was used to deconvolute the DNA frequency histograms to estimate the frequency of cells in different phases of the cell cycle, and those in apoptosis. The experiments were repeated several times, yielding essentially identical results.

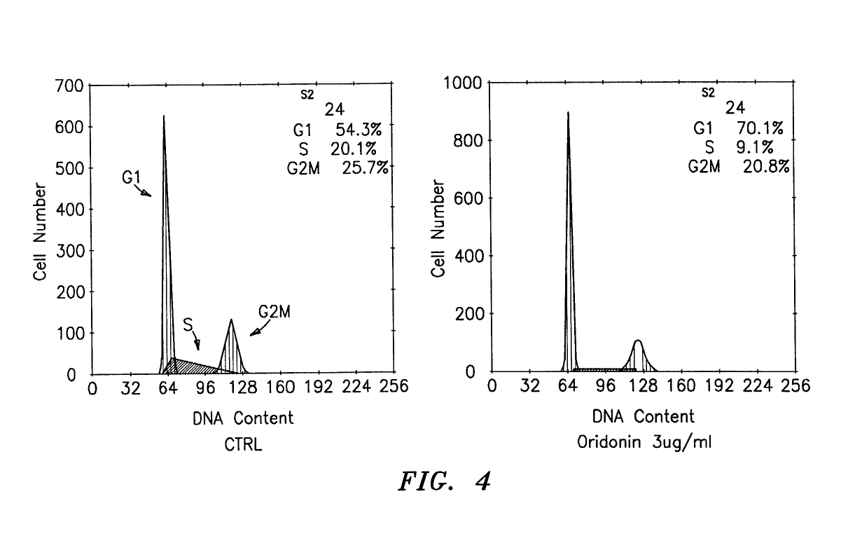

FIG. 4 displays the DNA histograms of LNCaP in the absence (“CTRL”) and in the presence of oridonin at 3 μg/ml after 24 hr of cell incubation. FIG. 4 shows that over 65% of the cells exposed to oridonin were in the G1 phase of the cell cycle at the end of the 24 hr incubation time, whereas only about 54% of the cells not exposed to oridonin were in the G1 phase after 24 hours. Data analysis revealed that the increase in the G1 phase was proportional to oridonin concentration.

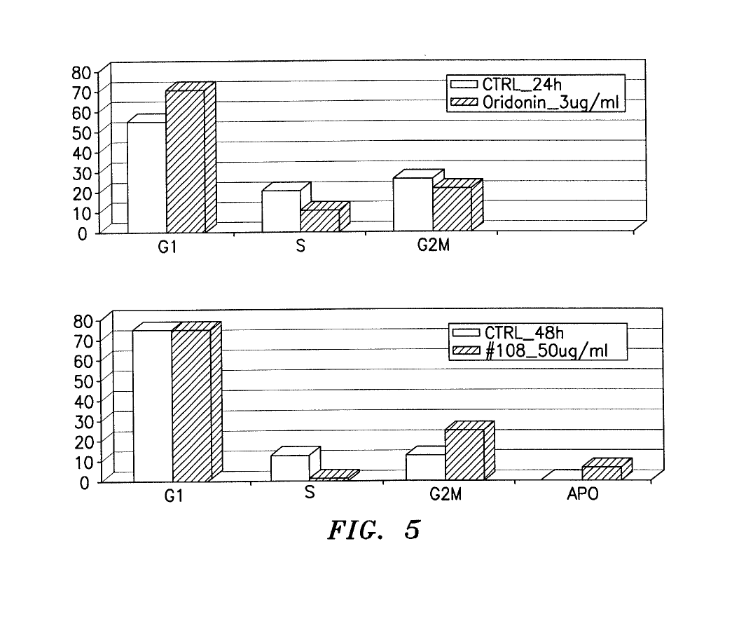

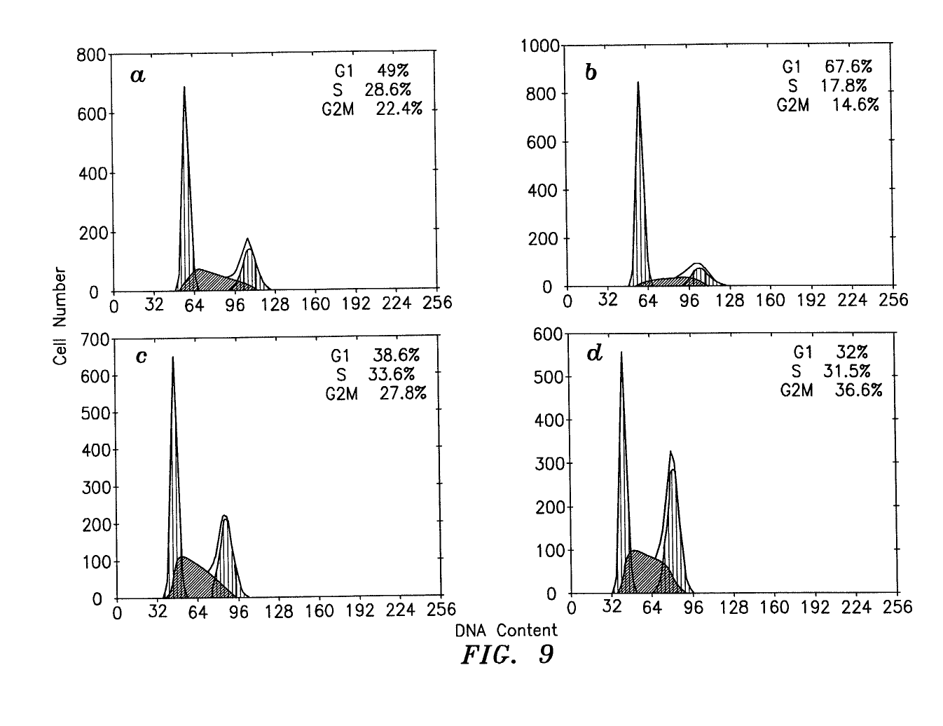

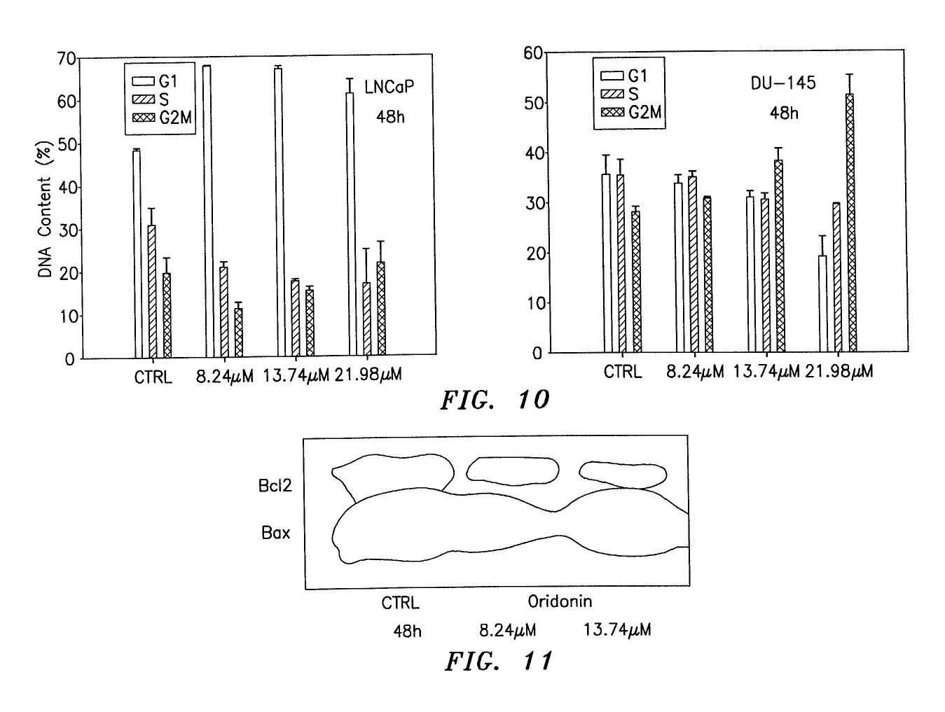

Similar measurements were conducted for lupulone. FIG. 5 summarizes the effect of oridonin and lupulone on G1, S, and G2M phases of LNCaP cell cycle. The data shows that over 25% more cells are arrested in the G1 phase of the cell cycle when exposed to 3 ug/ml of oridonin compared to when those cells are not exposed to oridonin. Similar to the data obtained in FIG. 5, FIGS. 9a and 9b also show the effect of oridonin on the cell cycle of LNCaP. As shown only about 49% of the cells not exposed to oridonin are arrested in the G1 phase, whereas about 68% of the cells exposed to 13.74 mM of oridonin are arrested in the G1 phase. Furthermore, FIG. 10 indicates that the effect on G1 phase arrest is dose-dependent on the amount of oridonin. As shown in FIG. 10, at about 8.20 uM-22 uM of oridonin, over 60% of the DU-145 cells are arrested in the G1 phase of the cell cycle.

As shown in FIG. 5, when cells are exposed to lupulone, an increase of about 100% of cells arrested in the G2M phase of the cell cycle is found in comparison to those cells not exposed to lupulone. Therefore, as a prolongation in either G1 or G2M phases leads to the suppression of LNCaP cell proliferation, both oridonin and lupulone are effective inhibitors of LNCaP cell proliferation.

### EXAMPLE 5

### The Effect of Oridonin on the DU-145 Cell Cycle

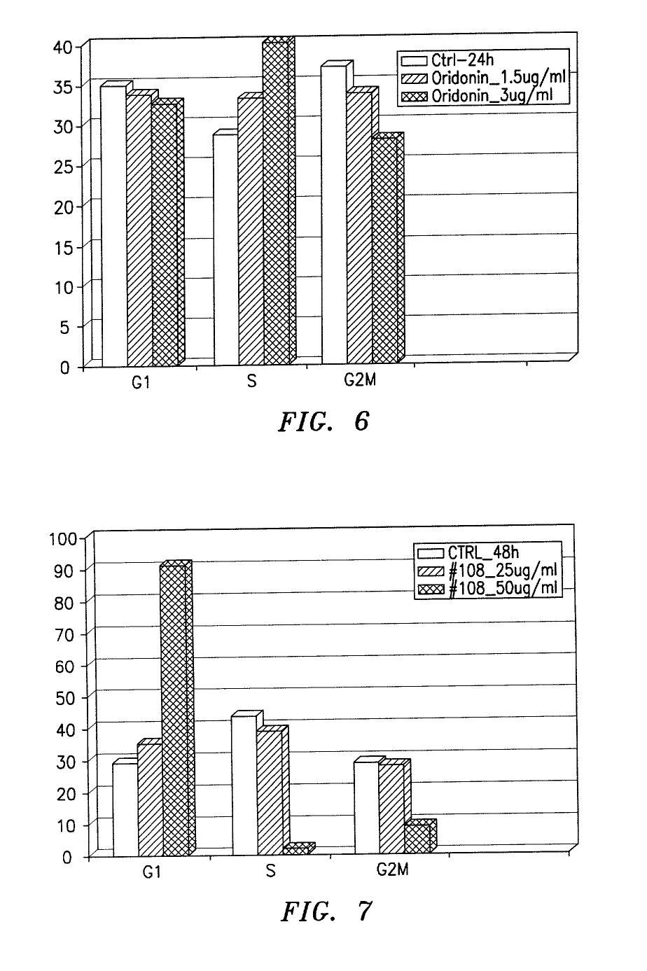

The protocol described in Example 2 was used to study the effect of oridonin on the hormone-independent prostate cancer cell line DU-145. As shown in FIG. 6, the number of cells arrested in the S phase are significantly greater when exposed to oridonin compared to when they are not. FIG. 6 shows an increase in the number of cells arrested in the S phase when exposed to 1.5 ug/ml of oridonin (about a 16% increase) and 3.0 ug/ml of oridonin (about a 30% increase) compared to the control. Similarly, FIG. 9 shows that when DU-145 cells are exposed to 13.74 mM of oridonin, about 37% of the cells are arrested in the G2M phase (FIG. 9d) as compared to the 28% of non-oridonin exposed cells arrested in the G2M phase (FIG. 9c). Furthermore, FIG. 10 indicates that the effect on G2M phase arrest is dose-dependent on the amount of oridonin. As shown in FIG. 10, at about 8.20 uM-22 uM of oridonin, about 30-55% of the DU-145 cells are arrested in the G2M phase of the cell cycle. Therefore, FIGS. 6, 9, and 10 show that oridonin prolongs the S phase of DU-145, thereby suggesting that oridonin suppresses DU-145 cell proliferation.

### EXAMPLE 6

### The Effect of Lupulone on the MCF-7 Cell Cycle

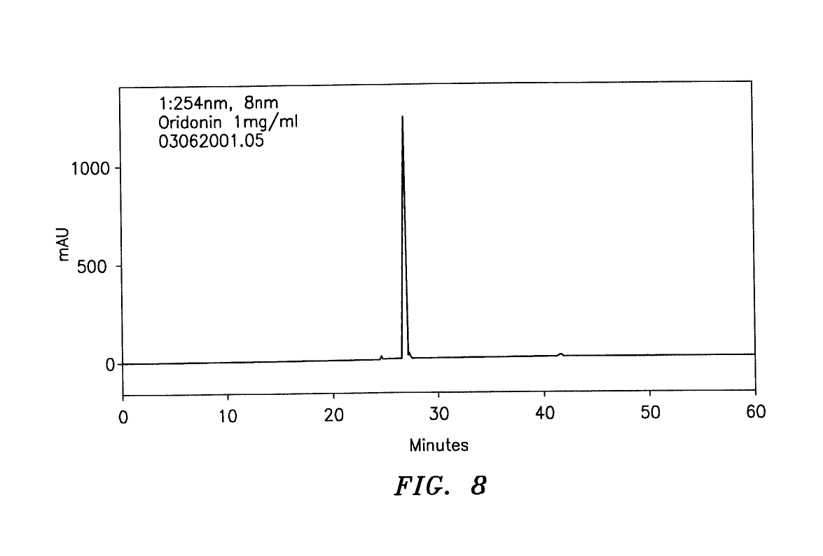

The protocol described in Example 2 was used to study the effect of lupulone on the hormone-dependent breast cancer cell line MCF-7. As shown in FIG. 7, the number of cells arrested in the G1 phase when exposed to 50 ug/ml of oridonin, is about 90%; when exposed to 25 ug/ml of oridonin, about 35% of the cells are arrested in the G1 phase; and when exposed to no oridonin, only about 28% of the cells are arrested in the G1 phase. The data in FIG. 7 show that lupulone at a concentration of 60 micromolar (25 microgram/milliliter), lupulone induced a G1 phase arrest very different from that observed in LNCaP (see FIG. 5) where the G2M phase was primarily affected. At the concentration of 120 micromolar (50 microgram/milliliter), a complete block of cell proliferation was achieved.

Summarizing the data from Examples 4-6, the antiproliferative property of oridonin is independent of androgen-receptor, as DU-145 and MCF-7 are equally sensitive to the effect of oridonin compared to LNCaP. Compounds, such as baicalein, baicalin, which are flavonoids found in Scutellaria Baicalensis Georgi, and DES have been shown to be more effective in inhibiting cell proliferation of LNCaP, and to a much lesser extent in suppressing DU-145. In a previous study, DES was found to be about 2.5 times more sensitive in inhibiting LNCaP than in inhibiting DU-145 as shown in FIG. 14. From this then, it is speculated that oridonin inhibits cell proliferation by a mechanism independent of androgen-receptors.

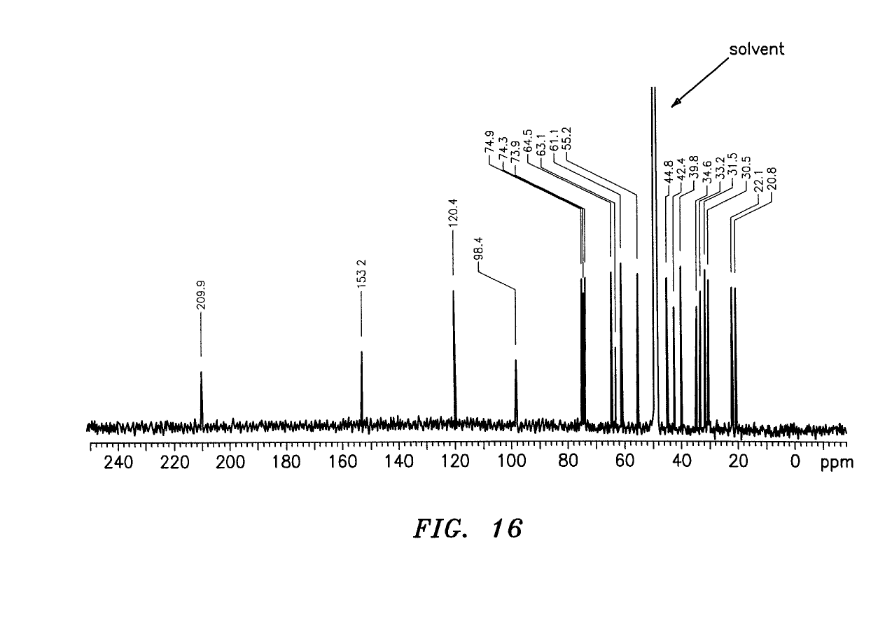

Furthermore, resembling PC SPES, oridonin led to a G1 cell cycle arrest in LNCaP and a G2M arrest in DU-145. DES, however, is known to cause a G2M phase arrest both in LNCaP and DU-145 cells as shown in FIG. 15. This difference in cell cycle modulation gives further support to the speculation that the mechanism of oridonin on prostate cancer cells is distinctive from the action of estrogenic compounds. As the mechanism behind estrogenic compounds is controlling cell growth, it is speculated that oridonin inhibits cell growth, not by proliferation, but by apoptosis, also known as cell death.

### EXAMPLE 7

### Bcl-2, p53 (Wild-Type), and Bax Protein Expression

Due to the variance in the modulation of the cell cycle between oridonin and DES, it is suspected that the antiproliferative activity of oridonin is primarily via the mechanism of enhancing pro-apoptotic genes and decreasing anti-apoptotic genes. It has been demonstrated in the past that the Bcl-2 and Bax gene products form homodimers and heterodimers and that the balance between the respective products determines the extent to which apoptosis is suppressed or promoted. The fact that oridonin reduced the ratio of Bcl-2/Bax suggests the apoptotic induction pathway of oridonin.

Growth of a cell population is a balance between cell proliferation and cell death. Therefore, the inhibitory effect by oridonin on LNCaP cells either affects the cells by reducing the rate of proliferation or by increasing the rate of cell death. One way of determining the mechanism behind the inhibitory effect of oridonin is by using changes in the Bcl-2 and Bax ratio. Bcl-2 is a onco-protogene known to stimulate cell proliferation and its expression is associated with the emergence of androgen-independent prostate tumors; whereas Bax is a cancer suppressor gene.

Another way of determining the mechanism behind oridonin's inhibitory effect is to determine the effect of oridonin on p53 protein expression. p53 is a tumor suppressor gene, and a transcription factor. Its protein levels tend to be extremely low in cells because of the rapid turnover of p53. p53 has long been linked to GI arrest and to inhibition of cell proliferation. Elevated expression of p53 promotes apoptotic death in many tumor cell lines including LNCaP.

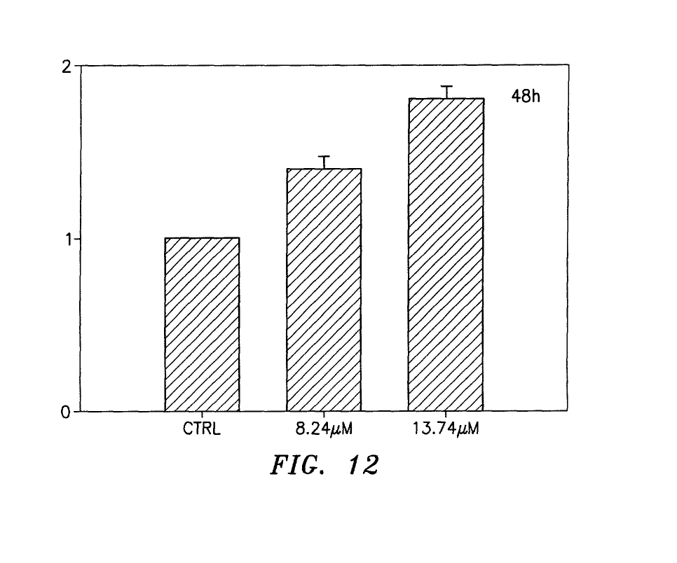

To determine the Bcl-2:Bax ratio in oridonin-incubated LNCaP cells, a Western blotting procedure was performed. LNCaP cells were prepared such that one sample contained was incubated in the absence of oridonin, another sample in 8.24 uM of oridonin, and another sample in 13.74 uM of oridonin. The samples were incubated for 48 hours. Equal amounts of protein (10 micrograms) from LNCaP cell lysates were applied to a 10% SDS/PAGE (sodium dodecylsulfate/polyacrylamide gel electrophoresis) gel, and were then transferred to a PVDF membrane. The blots were probed with one of two primary antibodies comprising 1:100 Anti-Bax YTH-2D2 (Lot#3148E0, R&D, MN USA), and 1:100 Bcl-2 Oncoprotein (Clone 124, No. M0887), followed by second antibody exposure of 1:1000 Anti-mouse IgG-HRP (se-2005, Santa Cruz Biotechnology, USA) for 1 hour at ______ temp. The immuno-binding signals were detected by the chemiluminescence method (ECL Western blotting system). The experiments were run in triplicates. As shown in FIG. 11, the ratio of Bax/Bcl-2 increased significantly in the presence of oridonin, thereby indicating that oridonin plays a role in up-regulating Bax protein expression, and in down-regulating the Bcl-2 expression.

To further verify the increase in Bax and the decrease in Bcl-2 proteins, and to determine the p53 protein levels induced by oridonin, flow cytometric measurement was performed to compare the results as obtained by Western blot. Following treatment with 3-9 μM of oridonin for 48 h, the LNCaP cells were trypsinized and washed with PBS. The cells were fixed in 1% formaldehyde in PBS on ice for 10 minutes and then permeabilized with 70% ethanol at −20° C. After fixation, the cells were rinsed with PBS, treated with a blocking solution containing 1% (w/v) bovine serum albumin (BSA) and 0.1% sodium azide in PBS (PBS-BSA) for 2 min at room temperature. The cells were subsequently added to a 100 μl aliquot of BSA/PBS solution containing 1:50 diluted primary antibody for Bcl-2, Bax (stained LNCaP) and p53. The mixtures were incubated at room temperature in the dark for 1 hr. The cells were subsequently washed twice with PBS-BSA buffer. The secondary antibody was added at 1:50 dilution for 30 minutes at room temperature in the dark, then counterstained for DNA by the addition of 1 ml of PI (phosphatidyl inositol) solution (final PI concentration 5 μg/ml) containing 100 μg/ml of RNAse A. Cellular fluorescence was measured with the ELITE ESP flow cytometer/cell sorter (Coulter Inc., Miami, Fla.) using the argon ion laser (emission at 488 nm). Fluorescence signals were collected using the standard configuration of the flow cytometer (green fluorescence for antibodies containing Bcl-2, Bax, and p53, and red fluorescence for DNA staining). 10,000 cells were analyzed per sample.

As shown in FIG. 12, p53 expression responds to the dose of oridonin used. Approximately 40% and 100% increases in p53 concentrations were observed at 8.2 uM and 13.74 uM of oridonin, respectively.

Table 3 summarizes the changes in Bax/Bcl-2 and p53/Bcl-2 ratios as measured by the flow cytometry method. Table 3 more specifically shows that approximately a 75% increase in the ratio of Bax/Bcl-2 was obtained in the presence of 8.24uM of oridonin. Also, as shown in Table 3, the accumulation of p53 in LNCaP increased in the presence of oridonin. The concentration ratio of p53 and Bcl-2 proteins obtained from flow cytometric measurement was also used to evaluate the pro-apoptotic property of oridonin on LNCaP. A 100% increase in the ratio of p53/Bcl-2 was observed in the presence of 8.24uM oridonin.

TABLE 3
Increase in Bax/Bcl-2 and p53/Bcl-2 in the presence of
oridonin measured by flow cytometry

| | Bax | Bcl-2 | Bax/Bcl-2 | p53 | p53/Bcl-2 |
|---|---|---|---|---|---|
| CTRL | 11.8 | 11.3 | 1 | 8.8 | 1 |
| 8.24 uM | 14.5 | 7.9 | 1.8 | 12.3 | 2.0 |
| oridonin | | | | | |

### EXAMPLE 8

### Apoptosis in LNCaP Cell Lines Caused by Oridonin Exposure

To determine apoptosis in cells, a quantitative assay for the detection of DNA breakage, using the terminal deoxyribonucleotide transferase (TdT) color reaction assay (TiterTACS, Trevigen, Gaithersburg, Md.), was employed. Cells were fixed in 1% ice-cold formaldehyde for 15 minutes and postfixed in 70% ethanol overnight at −20° C. DNA strand breaks were directly labeled with fluorescent (2′-deoxyuridine 5′-triphosphate) dUTP in the reaction catalyzed by exogenous terminal deoxynucleotidyltransferase using the APO-Direct kit (Phoenix Flow System, San Diego, Calif.) according to the protocol provided with the kit. The green (FITC) and red (PI) fluorescence intensities of cells subjected to labeling DNA strand breaks with the use of TdT were measured using a FACScan Flow Cytometer (Becton Dickinson Immunocytometry Systems, San Jose, Calif.). The data from 10⁴ cells per sample were collected, stored and analyzed using CELL Quest software (Becton Dickinson and Co.).

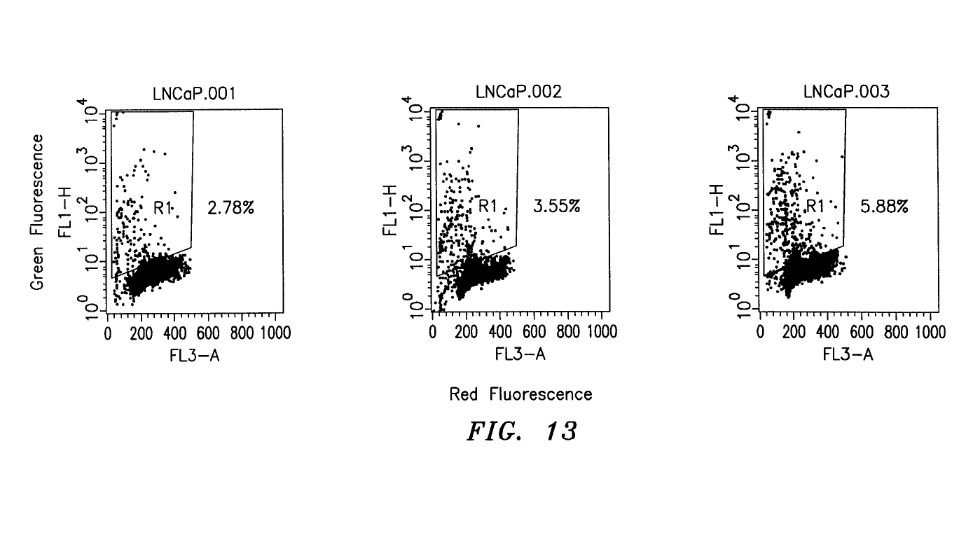

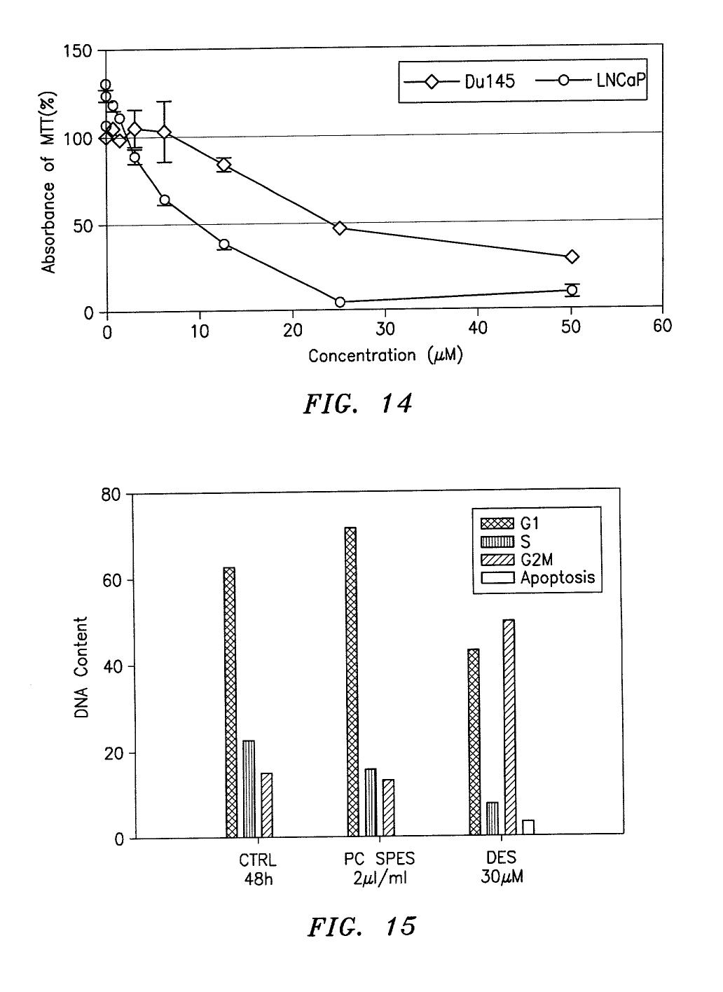

FIG. 13 shows a dose-responsive apoptosis of LNCaP induced by oridonin at concentrations of 0, 8.24 uM, and 13.74 uM at 48 hours. About 6% cell apoptosis of LNCaP was induced by oridonin at a concentration of 13.7uM on the ratio of p53/Bcl-2. A 100% increase was detected. p53 has long been linked to cell cycle G1 arrest and inhibition of cell proliferation. Elevated expression of p53 promotes apoptotic death in many tumor cell lines including LNCaP. Here, oridonin profoundly increased the concentration of p53 protein and prolonged G1 cell cycle in the LNCaP cell line.

The relationship between Bcl-2, Bax and p53 is a complex and important factor for the survival of cancer cells. Bax can be upregulated by p53. Alternatively p53 and Bcl-2 genes may be characterized, respectively as positive and negative regulators of cell death. Evidence indicates that p53-dependent cell death may be a downstream effect of Bcl-2 action. Likewise apoptosis may be suppressed by Bcl-2. The process may be induced by expression of the tumor suppressor gene p53.

Findings suggest that oridonin effectively synchronizes the down-regulation of Bcl-2 and up-regulation of Bax and p53 proteins. This synchronization action ensures the apoptotic cascade of prostate cancer cells.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

All cited patents, patent applications and other references are incorporated herein by reference in their entirety.

What is claimed is:

1. A composition for treating or preventing prostate cancer, breast cancer, colon cancer, lung cancer, or bladder cancer, the composition comprising a compound selected from the group consisting of oridonin, lupulone, bavachin, bavachalcone, bavachinin, bavachromene, their pharmaceutically acceptable salts or esters, their selectively substituted analogs, and combinations comprising at least one of the foregoing compounds.

2. The composition of claim 1, in an ingestible form.

3. The composition of claim 2, wherein the ingestible form is a powder, a capsule, or a tablet.

4. The composition of claim 1, in the form of a suppository.

5. The composition of claim 1, comprising a compound having the structure 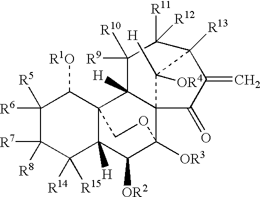
wherein R¹-R⁴ are each independently hydrogen, C₁-C₆ alkyl, or C₁-C₁₂ acyl, R⁵-R¹³ are each independently hydrogen or C₁-C₆ alkyl, and R¹⁴ and R¹⁵ are each independently C₁-C₆ alkyl, with the proviso that at least 4 of R⁵-R¹³ are hydrogen.

6. The composition of claim 5, wherein R¹-R⁴ are each independently hydrogen, methyl, ethyl, acetyl, or propionyl.

7. The composition of claim 5, wherein R⁵-R¹³ are each independently hydrogen, methyl, or ethyl.

8. The composition of claim 5, wherein R¹-R¹³ are hydrogen, and R¹⁴ and R¹⁵ are methyl.

9. The composition of claim 5, comprising an extract of Rabdosia rabescens.

10. The composition of claim 9, further comprising an extract of Humulus lupulus, Psoralea corylifolia L., or a combination thereof.

11. The composition of claim 1, comprising a compound having the structure 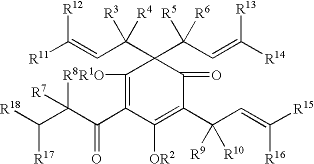
wherein R¹ and R² are each independently hydrogen, C₁-C₆ alkyl, or C₁-C₁₂ acyl; R³-R₁₀ are each independently hydrogen or C₁-C₆ alkyl with the proviso that at least four of R³-R¹⁰ are hydrogen; and R¹¹-R¹⁸ are each independently C₁-C₆ alkyl.

12. The composition of claim 11, wherein R¹ and R² are each independently hydrogen, methyl, ethyl, acetyl, or propionyl.

13. The composition of claim 11, wherein R³-R¹⁰ are each independently hydrogen, methyl, or ethyl.

14. The composition of claim 11, wherein R¹¹-R¹⁸ are each independently methyl or ethyl.

15. The composition of claim 11, wherein R¹-R¹⁰ are each hydrogen, and R¹¹-R¹⁸ are each methyl.

16. The composition of claim 11, comprising an extract of Humulus lupulus.

17. The composition of claim 16, further comprising an extract of Rabdosia rubescens, Psoralea corylifolia L., or a combination thereof.

18. The composition of claim 1, comprising a compound having the structure 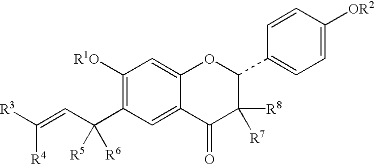
wherein R¹ and R² are each independently hydrogen, C₁-C₆ alkyl, or C₁-C₁₂ acyl; and R³-R⁸ are each independently hydrogen or C₁-C₆ alkyl with the proviso that at least two of R³-R⁸ are hydrogen.

19. The composition of claim 18, wherein R¹ and R² are each independently hydrogen, methyl, ethyl, acetyl, or propionyl.

20. The composition of claim 18, wherein R³-R⁸ are each independently hydrogen, methyl, or ethyl.

21. The composition of claim 18, wherein R³ and R⁴ are methyl.

22. The composition of claim 18, wherein R¹, R², and R⁵-R⁸ are hydrogen; and R³ and R⁴ are methyl.

23. The composition of claim 18, wherein R² and R⁵-R⁸ are hydrogen; and R¹, R³, and R⁴ are methyl.

24. The composition of claim 18, comprising an extract of Psoralea corylifolia L.

25. The composition of claim 24, further comprising an extract of Rabdosia rubescens, Humulus lupulus, or a combination thereof.

26. The composition of claim 1, comprising a compound having the formula 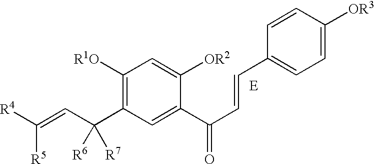
wherein R¹-R³ are each independently hydrogen, C₁-C₆ alkyl, or C₁-C₁₂ acyl; and R⁴-R⁷ are each independently hydrogen or C₁-C₆ alkyl.

27. The composition of claim 26, wherein R¹-R³ are each independently hydrogen, methyl, ethyl, acetyl, or propionyl.

28. The composition of claim 26, wherein R⁴-R⁷ are each independently AO hydrogen, methyl, or ethyl.

29. The composition of claim 26, wherein R¹-R³, R⁶, and R⁷ are hydrogen; and R⁴ and R⁵ are methyl.

30. The composition of claim 26, comprising an extract of Psoralea corylifolia L.

31. The composition of claim 30, further comprising an extract of Rabdosia rubescens, Humulus lupulus, or a combination thereof.

32. The composition of claim 1, comprising a compound having the structure 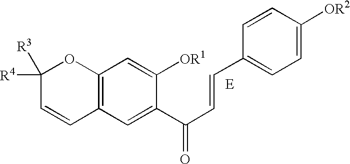
wherein R¹ and R² are each independently hydrogen, C₁-C₆ alkyl, or C₁-C₁₂ acyl; and R³ and R⁴ are each independently hydrogen or C₁-C₆ alkyl.

33. The composition of claim 32, wherein R¹ and R² are each independently hydrogen, methyl, ethyl, acetyl, or propionyl.

34. The composition of claim 32, wherein R³ and R⁴ are each independently hydrogen, methyl, or ethyl.

35. The composition of claim 32, wherein R¹ and R² are hydrogen; and R³ and R⁴are methyl.

36. The composition of claim 32, comprising an extract of Psoralea corylifolia L.

37. The composition of claim 36, further comprising an extract of Rabdosia rubescens, Humulus lupulus, or a combination thereof.

38. A composition for treating or preventing prostate cancer, breast cancer, colon cancer, lung cancer, or bladder cancer, the composition comprising an extract of a plant selected from the group consisting of Rabdosia rubescens, Humulus lupulus, Psoralea corylifolia L., and combinations comprising at least one of the foregoing plants.

39. A composition for treating or preventing prostate cancer, breast cancer, colon cancer, lung cancer, or bladder cancer, the composition comprising an extract of Rabdosia rubescens and an extract of a plant selected from the group consisting of Panax pseudo-ginseng Wall, Ganoderma lucidum Karst, Scutellaria baicalensis Georgi, Glycine max, Curcuma longa, and combinations comprising at least one of the foregoing plants.

40. The composition of claim 39, comprising:
about 2 to about 10 parts by weight of an extract of Rabdosia rubescens;
about 10 to about 40 parts by weight of an extract of Panax pseudo-ginseng Wall;
about 100 to about 500 parts by weight of an extract of Ganoderma lucidum Karst;
about 20 to about 100 parts by weight of an extract of Scutellaria baicalensis Georgi;
about 20 to about 100 parts by weight of an extract of Glycine max; and
about 20 to about 100 parts by weight of an extract of Curcuma longa.

41. The composition of claim 40, wherein the extract of Rabdosia rubescens comprises oridonin; wherein the extract of Panax pseudo-ginseng Wall comprises a gensenoside; wherein the extract of Scutellaria baicalensis Georgi comprises baicalin; wherein the extract of Glycine max comprises a soy flavonoid, a soy isoflavonoid, or both; and wherein the extract of Curcuma longa comprises curcumin.

42. A composition for treating or preventing prostate cancer, breast cancer, colon cancer, lung cancer, or bladder cancer, the composition comprising an extract of Humulus lupulus and an extract of a plant selected from the group consisting of Panax pseudo-ginseng Wall, Ganoderma lucidum Karst, Scutellaria baicalensis Georgi, Glycine max, Curcuma longa, and combinations comprising at least one of the foregoing plants.

43. The composition of claim 42, comprising:
about 2 to about 10 parts by weight of an extract of Humulus lupulus;
about 10 to about 40 parts by weight of an extract of Panax pseudo-ginseng Wall;
about 100 to about 500 parts by weight of an extract of Ganoderma lucidum Karst;
about 20 to about 100 parts by weight of an extract of Scutellaria baicalensis Georgi;
about 20 to about 100 parts by weight of an extract of Glycine max; and
about 20 to about 100 parts by weight of an extract of Curcuma longa.

44. The compositions of claim 43, wherein the extract of Humulus lupulus comprises lupulone; wherein the extract of Panax pseudo-ginseng Wall comprises a gensenoside; wherein the extract of Scutellaria baicalensis Georgi comprises baicalin; wherein the extract of Glycine max comprises a soy flavonoid, a soy isoflavonoid, or both; and wherein the extract of Curcuma longa comprises curcumin.

45. A method of treating prostate cancer, breast cancer, colon cancer, lung cancer, or bladder cancer in an individual in need thereof, comprising administering a therapeutically effective amount of the composition of claim 1.

46. A method of treating prostate cancer, breast cancer, colon cancer, lung cancer, or bladder cancer in an individual in need thereof, comprising administering a therapeutically effective amount of the composition of claim 39.

47. A method of treating prostate cancer, breast cancer, colon cancer, lung cancer, or bladder cancer in an individual in need thereof, comprising administering a therapeutically effective amount of the composition of claim 42.
